(19) United States
(12) Patent Application Publication
(10) Pub. No.: US 2021/0393452 A1
(43) Pub. Date: Dec. 23, 2021

(54) FEMININE PAD WITH LARGE PETAL DESIGN

(71) Applicants: Katrina Marie Arceo-Yap, Santa Clarita, CA (US); Audrey Rose Adrineda, Santa Clarita, CA (US); Claire Agcaoili Malabanan, Santa Clarita, CA (US)

(72) Inventors: Katrina Marie Arceo-Yap, Santa Clarita, CA (US); Audrey Rose Adrineda, Santa Clarita, CA (US); Claire Agcaoili Malabanan, Santa Clarita, CA (US)

(21) Appl. No.: 17/304,599

(22) Filed: Jun. 23, 2021

Related U.S. Application Data
(60) Provisional application No. 62/705,348, filed on Jun. 23, 2020.

(51) Int. Cl. A61F 13/472; A61F 13/56

(52) U.S. Cl. CPC A61F 13/472; A61F 2013/4706; A61F 13/5616

(57) ABSTRACT

A feminine hygiene pad with a large petal protrusion at an aft end of the pad. The feminine pad is used to prevent leakage from seeping around the buttocks region, especially while seated or in a supine position. The invention improves over existing pads due to the large petal shape for coverage on the buttocks portion and on the material used for absorbency.

### CROSS-REFERENCE TO RELATED APPLICATION

This application claims the benefit of priority of U.S. provisional application No. 62/705,348 filed Jun. 23, 2020, the contents of which are herein incorporated by reference.

### BACKGROUND OF THE INVENTION

The present invention relates to feminine hygiene pads, and more particularly feminine hygiene pads to prevent leakage.

Other feminine hygiene pads are narrow and do not take into account other positions that the wearer may assume throughout the day, such as sitting or being supine. On current products, when the user is in the supine or sitting position, fluids may leak out from the feminine pad and may be absorbed onto clothing, bedding, or other areas where the user may be in a supine or sitting position.

Accordingly, there is a need for an improved feminine hygiene pad that prevents leakage. The invention improves due to the large round-like petal shape of the coverage on the buttocks portion and on the material used for absorbency. The feminine pad is used to prevent leakage from seeping around the buttocks region.

### SUMMARY OF THE INVENTION

In one aspect of the present invention, a feminine hygiene pad is disclosed. The feminine hygiene pad includes a plurality of layers including an outer liquid impermeable base layer, an inner liquid permeable layer, and an absorbent layer interposed between the outer liquid impermeable base layer and the inner liquid permeable layer. The plurality of layers are formed having a narrow forward end and an aft, petal shaped end. The narrow forward end is dimensioned to extend from a labia majora to a point proximal the anus, when worn. The narrow forward end diverges outwardly from a longitudinal centerline of the feminine hygiene pad to define the aft petal shaped end, The aft petal shaped end terminates at a buttocks region of the wearer, when worn.

In some embodiments, a pair of wings laterally diverge from an intermediate portion of the feminine hygiene pad. The pair of wings are adapted to at least partially wrap around an intermediate area of an undergarment between a user's legs, when worn.

In some embodiments, an adhesive is disposed on an outer surface of the base layer. A removable backing layer covers the adhesive.

A peripheral edge of the plurality of layers may be joined. A longitudinal length of the feminine hygiene pad is on the order of 47 cm long. A longitudinal length of the narrow front end is on the order of 9 cm. A width of the aft, petal shaped end is on the order of 26 cm wide.

In other aspects of the invention, a method of feminine hygiene protection is disclosed. The method includes providing a feminine hygiene pad having an outer liquid impermeable base layer, an inner liquid permeable layer, and an absorbent layer interposed between the outer liquid impermeable base layer and the inner liquid permeable layer. The plurality of layers are formed having a narrow forward end and an aft, petal shaped end. The narrow forward end is dimensioned to extend from a labia majora to a point proximal the anus, when worn. The narrow forward end divergesg outwardly from a longitudinal centerline of the feminine hygiene pad to define the aft petal shaped end. The aft petal shaped end terminates at a buttocks region of the wearer, when worn. The method includes applying an outer surface of the base layer to an inner surface of a garment worm on the user's buttocks and urogenital area.

In some embodiments, the method includes removing a backing layer covering an adhesive applied to an outer surface of the outer liquid impermeable base layer. The adhesive is then applied to the inner surface of the garment.

The method may also include holding the forward end of the feminine hygiene pad and extending the aft, petal shaped end over a posterior end of the garment.

In some embodiments, the method also includes wrapping a pair of wings laterally extending from an intermediate portion of the feminine hygiene pad around an intermediate area of the garment positioned between a user's legs, when worn.

The method may also include pulling the garment upwards around the buttocks and urogenital area.

These and other features, aspects and advantages of the present invention will become better understood with reference to the following drawings, description and claims.

### BRIEF DESCRIPTION OF THE DRAWINGS

FIG. 1 is a perspective view of the feminine hygiene pad.

FIG. 2 is a rear view of the feminine hygiene pad, shown in use.

FIG. 3 is an exploded view of the feminine hygiene pad.

FIG. 4 is a section view of the feminine hygiene pad taken along line 4-4 in FIG. 1.

### DETAILED DESCRIPTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, embodiments of the present invention provide an apparatus and method for an improved feminine hygiene pad that prevents leakage, particularly when the user is in a seated or a supine position.

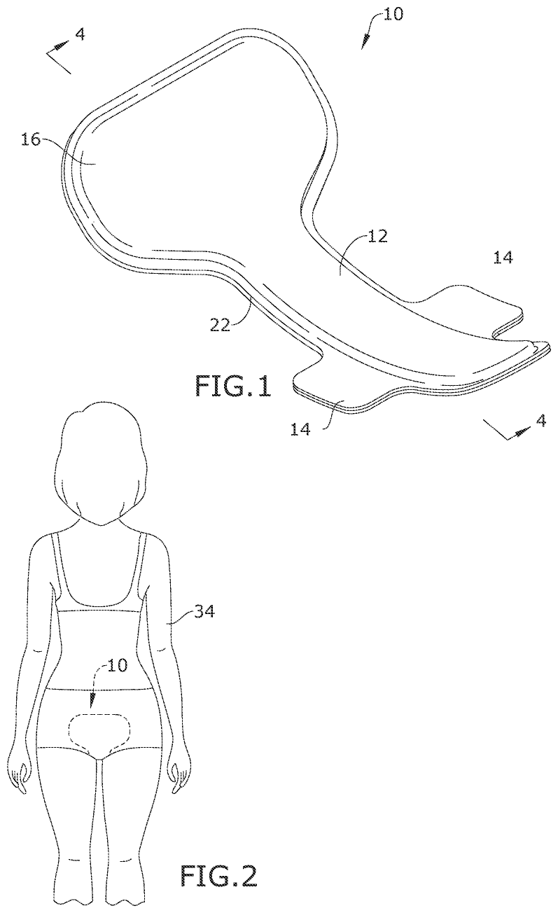

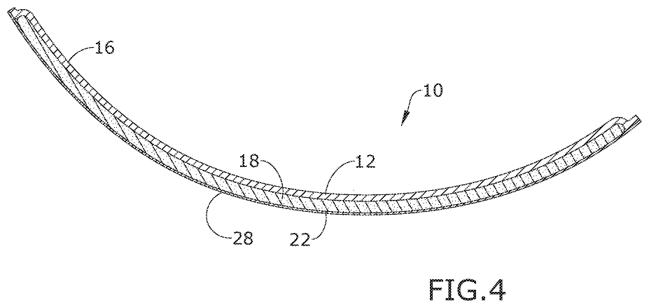

As seen in reference to FIGS. 1-4, the improved feminine hygiene pad 10 is a generally pendulum shaped pad. A forward end 12 of the pad 10 includes a thin shaped area extending from the labia majora past the anus. The forward end has a width dimensioned to cover the genitalia and fit between the wearer's 34 legs. The forward end 12, then widens, or diverges outwardly from a longitudinal centerline of the pad 10, forming a petal shaped 13 area covering the buttocks region. Small wings 14 may extend laterally outwardly from an intermediate portion of the feminine hygiene pad 10 to secure the feminine hygiene pad 10 to a center of the wearer's underwear. The aft end 16 has a large petal shaped area 16 to shield the buttocks from menstruation overflow, especially when the user is in a sitting or supine position.

The feminine hygiene pad 10 is formed of a quick fluid absorbent material and the petal shaped area 16 covers the buttocks region where menstrual fluids may seep to and around with a conventional feminine hygiene pad. The feminine hygiene pad 10 of the present invention prevents fluids from leaking onto clothing, furniture and bedding from a posterior end of the feminine hygiene pad 10.

Other feminine hygiene pads in the art do not work well due to the generally rectangular shape of the pad region that terminates at the buttocks region. These conventional feminine hygiene pads do not provide an enlarged area at the aft end of the pad where fluids may be absorbed on to. The present invention improves upon the prior art due to the large round-like petal shaped area 16 that provides coverage on the buttocks portion and on the material used for absorbency.

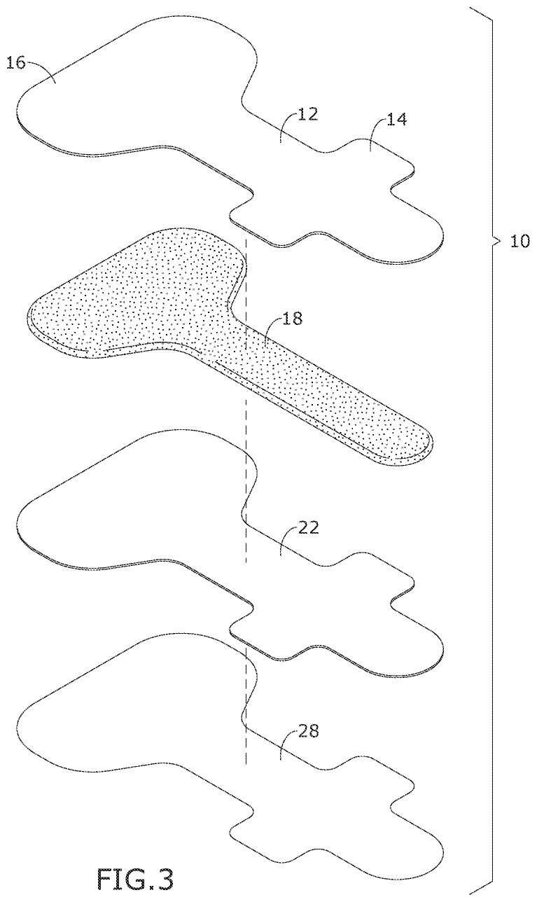

As best seen in reference to FIG. 3, the feminine hygiene pad 10 is comprised of a plurality of layers including a liquid impermeable base 22, an absorbent layer 18, which may include a gel or gel forming material, and a cotton or quilted inner liquid permeable layer 12, such as a soft cotton mesh. An adhesive layer may be applied to an outer surface of the base layer 22, for securing the feminine hygiene pad 10 to an inner surface the user's 34 garments. A releasable backing layer 28 may also be provided to cover the adhesive when the feminine hygiene pad 10 is not in use.

The base layer 22 will preferably be formed of a liquid impermeable biodegradable material. The middle absorbent layer 18 may formed of an absorbent cotton with an odor and a fluid absorbing gel forming material embedded with the absorbent layer 18. The fluid absorbing gel forming material may be a powder or a pelletized composition. The top layer 12, positioned proximal to the body surface, may be formed a soft material with quilted design for quicker absorption. The plurality of layers may be joined around a peripheral edge with an adhesive, a stitch, a weld, or other bond to retain its shape and structure.

### Non-Limiting Embodiments of the Pad May Include:

1. An overall length of the feminine hygiene pad 10 that may be on the order of 47 cm long.
2. A front portion 11 of the pad is rounded and on the order of 9 cm in length.
3. The wing 14 width across the feminine hygiene pad10 is on the order of 19 cm.
4. The rounded back or petal shaped area 13 of the pad 10 is on the order of 26 cm wide.

In a representative, non-limiting embodiment of the improved feminine hygiene pad 10, the full longitudinal length of the pad is approximately 47 cm. The front portion of the pad is 9 cm wide. The wing width is on the order of 14 cm wide. Each of the dimensions may be sized according to anthropometric measurements of the user (small, medium, large, etc.).

As seen in reference to FIG. 2, the feminine pad 10 is dimensioned to cover both the front, genital region, and the buttocks region of the wearer 34. As menstrual fluid is excreted onto the pad 10, the mesh top layer 12 will wick the fluids, drawing the fluids down into the absorbent layer 18 of the pad. The cotton will further pull the liquid down to the base layer 22, which may include absorbent beads that will trap the fluid creating a gel that will also capture the odor of the fluid.

When the user is in either the sitting or supine position, the petal shaped portion 16 of the feminine hygiene pad 10 will protect from seeping or leakage of fluids as the body excretes fluids, which tend to seep or drip to the buttocks due to gravity. The unique shape of the feminine hygiene pad 10 will provide full protection from seeping and leakage as the user may be in various positions from sitting to supine.

The larger, petal shaped end 16 is positioned to cover the buttocks to absorb excess menstrual flow, thus protecting clothes and bed linens when used at night during menstrual period. The feminine hygiene pad 10 may be utilized during post-surgical interventions, such as post-hemorrhoidectomy, post-partum delivery, post-caesarian section, post-prostatectomy, hysterectomy, and other procedures.

The following steps describe a method of use for the feminine hygiene pad:

1) Place the base layer 22, or backing layer 28 of the feminine hygiene pad 10 on the front/anterior end of the panty/undergarment.

2) Hold the top of the feminine hygiene pad 10 firmly and gently unroll the pad from the front of the panty/undergarment to extend the petal shaped end 16 over the back/posterior end of the panty in one motion.

3) Unfold the wings 14 of the feminine hygiene pad 10 and secure under the middle of the panty/undergarment.

4) Unfold the petal shaped area 16 of the feminine hygiene pad 10 and secure to the back/posterior side of the panty/undergarment.

5) Once pad is secure to panty/undergarment wearer may pull u the panty/undergarment to be worn in place.

When packaged, the aft, petal shaped end 16 may be folded over the forward end, with the lateral extents of the aft, petal shaped end 16 folded inwardly along a longitudinal centerline of the feminine hygiene pad 10. The feminine hygiene pad 10 may be carried in a protective wrapper.

It should be understood, of course, that the foregoing relates to exemplary embodiments of the invention and that modifications may be made without departing from the spirit and scope of the invention as set forth in the following claims.

What is claimed is:

1. A feminine hygiene pad, comprising:
a plurality of layers including an outer liquid impermeable base layer, an inner liquid permeable layer, and an absorbent layer interposed between the outer liquid impermeable base layer and the inner liquid permeable layer; and
the plurality of layers are formed having a narrow forward end and an aft, petal shaped end, the narrow forward end is dimensioned to extend from a labia majora to a point proximal an anal area of a user, when worn, the narrow forward end diverging outwardly from a longitudinal centerline of the feminine hygiene pad to define the aft petal shaped end, the aft petal shaped end terminating at a buttocks region of the user, when worn.

2. The feminine hygiene pad of claim 1, further comprising:
a pair of wings laterally diverging from an intermediate portion of the feminine hygiene pad, the pair of wings adapted to at least partially wrap around an intermediate area of an undergarment between a user's legs, when worn.

3. The feminine hygiene pad of claim 2, further comprising:
an adhesive disposed on an outer surface of the base layer; and
a backing layer covering the adhesive.

4. The feminine hygiene pad of claim 3, wherein a peripheral edge of the plurality of layers are joined.

5. The feminine hygiene pad of claim 4, wherein a longitudinal length of the feminine hygiene pad is on the order of 47 cm long.

6. The feminine hygiene pad of claim 5, where a longitudinal length of the narrow forward end is on the order of 9 cm.

7. The feminine hygiene pad of claim 6, wherein a width of the aft, petal shaped end is on the order of 26 cm wide.

8. A method of feminine hygiene protection, comprising:
providing a feminine hygiene pad having a plurality of layers comprising an outer liquid impermeable base layer, an inner liquid permeable layer, and an absorbent layer interposed between the outer liquid impermeable base layer and the inner liquid permeable layer; the plurality of layers are formed having a narrow forward end and an aft, petal shaped end, the narrow forward end is dimensioned to extend from a labia majora to a point proximal an anal area of a user, when worn, the narrow forward end diverging outwardly from a longitudinal centerline of the feminine hygiene pad to define the aft petal shaped end, the aft petal shaped end terminating at a buttocks region of the user, when worn; and
applying an outer surface of the base layer to an inner surface of a garment worm on a buttocks and a urogenital area of the user.

9. The method of claim 8, further comprising:
removing a backing layer covering an adhesive applied to an outer surface of the outer liquid impermeable base layer; and
adhering the adhesive to the inner surface of the garment.

10. The method of claim 8, further comprising:
holding the forward end of the feminine hygiene pad, and extending the aft, petal shaped end over a posterior end of the garment.

11. The method of claim 10, further comprising:
wrapping a pair of wings laterally diverging from an intermediate portion of the feminine hygiene pad around an intermediate area of the garment positioned between a user's legs, when worn.

12. The method of claim 10, further comprising:
pulling the garment upwards around the buttocks and urogenital area.
